(19) United States
(12) Patent Application Publication
(10) Pub. No.: US 2005/0113300 A1
(43) Pub. Date: May 26, 2005

(54) Method for use of pharmacological agent in the diagnosis of obsessive-compulsive disorder

(76) Inventor: Tomas Eriksson, Billdal (SE)

Correspondence Address: ORUM & ROTH, 53 W. JACKSON BLVD, CHICAGO, IL, 60604, US

(21) Appl. No.: 10/959,520

(22) Filed: Oct. 6, 2004

Related U.S. Application Data
(63) Continuation of application No. 09/980,023, filed on Nov. 28, 2001, now abandoned.

(30) Foreign Application Priority Data
Jun. 1, 1999 (SE) 9902026-5

(51) Int. Cl. A61K 38/25

(52) U.S. Cl. 514/12

(57) ABSTRACT

The invention relates to the use of a pharmacological agent for diagnosis of OCD; Obsessive-compulsive disorder. The invention makes it possible for patients suffering from OCD to obtain a more accurate diagnosis and an indication on the intensity of the disorder.

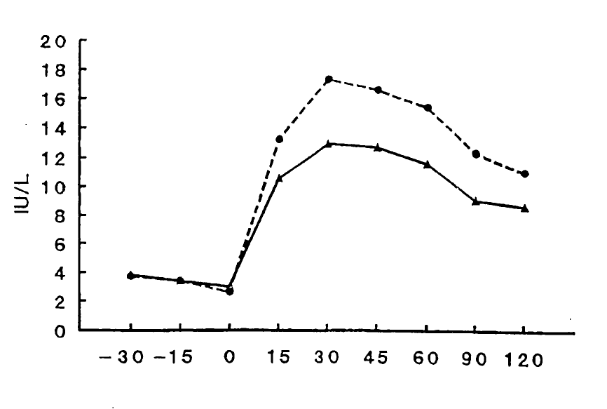

### TECHNICAL FIELD

The present invention relates to the use of a pharmacological agent in the diagnosis of obsessive-compulsive disorder (OCD).

### THE BACKGROUND OF THE INVENTION

Obsessive-Compulsive Disorder

OCD is a chronic psychiatric disease where the main symptoms are constituted by the patient having compulsive thoughts that he/she can not fend off and which often in a painful and destructive way prevents the person from thinking of other things or that the patient in a compulsive manner performs ritual acts that block the possibility for the person to devote herself or himself to other activities. The disorder is usually chronic and often so serious that the patient is completely or partially incapacitated.

The disorder is described and defined in detail in The Diagnostic and Statistical Manual of Mental Disorders, fourth edition (DSM-IV) published by the American Psychiatric Association in 1994.

State of the Art in the Diagnosis of OCD

In the clinic the disorder is diagnosed on the basis of information given by the patient on the present symptoms. At the present state of the art of science no objective method for the diagnosis of the disorder or for the estimation of its severity is available.

Physiological Regulation of Androgenic Hormones Under Normal Conditions (i.e. Without Influence of Drugs)

A hormone—gonadotropin releasing hormone (GnRH) is produced in a certain part of the brain. GnRH—in its turn—stimulates the production of so called gonadotropins in the pituitary (at the bottom of the brain). In man the gonadotropins are the luteinizing (LH) hormone and the follicle-stimulating hormone (FSH). These hormones are released to the blood and transported to the testes and the adrenal glands (of the male) and to the ovaries and the adrenal glands (of the female). In these glands the gonadotropins stimulate the synthesis of several different hormones among them the so called androgens (the male sex hormones) of which testosterone is the most common.

The androgenic hormones are released to the blood from the glands in which they are produced. They are transported with the blood to different organs where they exert their various actions. One of these organs is the brain. The androgenic hormones exert their effects in the brain by binding to and stimulating so called receptors in certain parts of the brain. The determining factor for how strong androgenic activity that will be exerted, is on one hand the amount of androgenic hormone in the blood, on the other the density and sensitivity of the receptors to which the androgenic hormones bind. The androgenic activity may thus be high, both at a high concentration of androgenic hormone in the blood, as well as in case of a high density and/or sensitivity of the androgenic receptors.

The synthesis of androgenic hormones is normally subjected to a so called “feed-back” regulation. If the androgenic activity in the brain is high, a compensating decrease in the release of gonadotropins takes place with an accompanying reduction of the production of androgenic hormones. At a high androgenic activity in the brain owing to a high density and/or sensitivity of the receptors (and not due to the content of androgenic hormones in the blood being high), the compensating feed-back-regulation may lead to a decreased production of androgenic hormones causing abnormally low level, without this in itself being a sign that the androgenic activity being low; it may still be high (if the compensation has not been sufficient) or normal (if compensation has been sufficient).

So-Called GnRH-Analogues

These are substances that in their effects resemble the endogenously produced GnRH (gonadotropin releasing hormone), that is they stimulate the release of the gonadotropins from the pituitary to the blood. The GnRH-analogues are used as pharmacological agents for two purposes. First, they are used to reduce the androgenic activity for example in cases of cancer of the prostate. This is achieved by a down-regulation in the sensitivity in those receptors on which endogenous GnRH and analogues of GnRH act. Such a down-regulation is established after treatment during a certain period of time with a subsequent inhibition of the synthesis of androgens.

Secondly, they are used in the diagnosis of certain somatic disorder by means of the so-called GnRH-test (see below).

The GnRH-Test

A deviant sensitivity and/or density in the GnRH receptors in CNS is present in certain endocrine disorders. Such deviations could be investigated with the so-called GnRH-test in which a small amount of an analogue of GnRH is injected intravenously. Blood samples are collected with short time intervals after the injection in which the concentrations of LH and/or FSH are determined. Under normal conditions, an increased release of LH and FSH is seen in healthy subjects after an injection of an analogue of GnRH. In various endocrine diseases, deviations in the release of LH and/or FSH after the injection with a analogue of GnRH is seen. By this procedures, a deviant sensitivity in the GnRH-receptors could be demonstrated. The use of this diagnostic method is, for example, described in Hormone Res. 6:177-191 (1975), P. Franchimont et al.

The Technical Problem

The objective of the present invention is to provide a pharmaceutical composition which enables the diagnosis and the assessment of the severity of the psychiatric disorder OCD by the use of a diagnostic test with this composition.

The Solution

For this object, the invention is characterised in that the composition comprises at least one substance within the group GnRH-analogue for the production of a pharmacological agent for the diagnosis of obsessive-compulsive disorder (OCD).

### DESCRIPTION OF A DRAWING

The invention will be described with reference to a drawing which is enclosed and which demonstrates the results from investigations of patients and healthy control subjects.

### DETAILED DESCRIPTION OF PERFORMED EXAMPLES OF THE INVENTION

The diagnostic method, described below, has its intellectual basis in a combination of observations made in contacts with patients on a specialised psychiatric clinic in Göteborg, and established scientific facts. In addition to that, the method is based on a scientific experiment.

To sum up, it has reference to the following observations and facts.

1. It was recently discovered that the disorder OCD could effectively be treated with a long-acting analogue of the gonadotropin-releasing hormone (GnRH). That observation demonstrates that the androgenic activity in the central nervous system (CNS), for some reason, is increased in OCD. Since the concentration of androgenic hormones in blood not has been shown to be increased in OCD, the sensitivity and/or the density of those receptors in CNS, which are stimulated by the androgenic hormones, must be increased. Such an increased hormonal activity causes, according to well-known physiological principles, by a feed-back regulation, a decrease in the release of the stimulating hormone. In the present case, a feed-back regulation might be mediated via GnRH by a decreased release of this hormone. Such a decreased release should, according to well-known physiological principles, cause an increase in the sensitivity in the GnRH-receptors in CNS.

2. In a scientific experiment six patients, all suffering from a severe form of OCD, have been examined by the so-called GnRH-test. For comparison, five healthy controls were examined. This experiment showed that the release of LH, after the infection of an analogue of GnRH was more pronounced in the patients suffering from OCD than in the control subjects. This finding strengthens the hypotheses that there is an increased sensitivity in the GnRH-receptors in patients with OCD and it shows that the GnRH-test, used within somatic medical care, could be used in the diagnosis of this psychiatric disorder.

The drawing shows, as a graph, the result of the GnRH-test in patients suffering from the disorder OCD and in a comparison group of healthy control subjects. In the graph, the abscissa gives the time in minutes and the ordinate the concentration of luteinizing hormone (LH) in blood. The graph show the arithmetic mean of the concentration of luteinizing hormone in patients suffering from the disorder OCD (broken line)(n=6) and in healthy control subjects (solid line) (n=5). The collection of blood samples started with a first blood sample which 15 min later followed by a second sample. At the time point 0 (according to the graph) still one blood sample was collected and 0.1 mg Relefact®. LH-RH, which is commercially available from Hoechst Marion Roussel, was injected intravenously. After that, blood samples were collected 6 times with intervals of 15, 30, 45, 60, 90, and 120 min. The blood samples were analysed with a radioimmunologic technic.

In a statistical assessment by means of a analysis of variance for repeated measures the results obtained from the group of patients have shown to be statistically different from the control group (F=5.6; p<0.05). Thus, the difference between the two groups is significant. Furthermore, individual differences within the patient group indicate that patient who show a high sensitivity (high concentration of LH in the blood) in this diagnostic test also show a higher intensity of the disorder. Thus, the pharmacological agent described above, could be use to improve the diagnosis of OCD and thus make it easier for people suffering from OCD to receive an adequate treatment.

1. (canceled)

2. Method according to claim 5, wherein the pharmacological agent is administered by means of intravenous injection.

3. Method according to claim 5, wherein the pharmacological agent is administered by means of a nasal spray.

4. (canceled)

5. Method of diagnosing an obsessive compulsive disorder comprising the steps of:
administering to a patient a pharmacological agent comprising at least one substance within the group GnRH analogues that release gonadotropins from the pituitary.

6. Method of assessing the severity of an obsessive disorder comprising the steps of:
administering to a patient with obsessive compulsive disorder a pharmacological agent comprising at least one substance within the group GnRH analogues that release gonadotropins from the pituitary.

7. Method according to claim 6, wherein the pharmacological agent is administered by means of intravenous injection.

8. Method according to claim 6, wherein the pharmacological agent is administered by means of a nasal spray.

9. The method of claim 5 comprising the additional steps of:
collecting at least one blood sample from the patient within a short time interval after the administration of the pharmacological agent,
determining the concentration of LH and/or FSH of the at least blood sample collected after the administration of the pharmacological agent,
comparing the concentration of LH and/or FSH of the at least one blood sample collected after the administration of the pharmacological agent to a blood concentration of LH and/or FSH before the administration of the pharmacological agent.

10. The method of claim 9 wherein the pharmacological agent is administered via an injection.

11. The method of claim 9 wherein the pharmacological agent is administered via a nasal spray.

12. The method of claim 9 comprising the additional steps of:
collecting at least one blood sample from the patient before the administration of the pharmacological agent, and
determining the concentration of LH and/or FSH of the blood sample collected before the adminstration of the pharmacological agent.

13. The method of claim 12 wherein the pharmacological agent is administered via an injection.

14. The method of claim 12 wherein the pharmacological agent is administered via a nasal spray.

15. The method of claim 6, comprising the additional steps of:
collecting at least one blood sample from the patient within a short time interval after the administration of the pharmacological agent,
determining the concentration of LH and/or FSH of the at least one blood sample collected after the administration of the pharmacological agent,
comparing the concentration of LH and/or FSH of the at least one blood sample collected after administration of the pharmacological agent to a blood concentration of LH and/or FSH before the administration of the pharmacological agent,
determining the severity of the obsessive compulsive disorder using the blood concentration of LH and/or FSH of the at least one sample collected after the administration of the pharmacological agent.

16. The method of claim 15 wherein the pharmacological agent is administered via an injection.

17. The method of claim 15 wherein the pharmacological agent is administered via a nasal spray.

18. The method of claim 15 comprising the additional steps of:
collecting at least one blood sample before the administration of the pharmacological agent, and
determining the concentration of LH and/or FSH of the blood sample collected before the administration of the pharmacological agent.

19. The method of claim 18 wherein the pharmacological agent is administered via an injection.

20. The method of claim 18 wherein the pharmacological agent is administered via a nasal spray.

21. The method of claim 9 wherein the time interval is less than 90 minutes.

22. The method of claim 9 comprising the additional step of:
analyzing the blood samples with a radioimmunologic technique.
